(19) United States
(12) Patent Application Publication
(10) Pub. No.: US 2018/0271695 A1
(43) Pub. Date: Sep. 27, 2018

(54) BODY-PART COOLING APPARATUS

(71) Applicant: Patrick Pace, Cheyenne, WY (US)

(72) Inventor: Patrick Pace, Cheyenne, WY (US)

(21) Appl. No.: 15/937,835

(22) Filed: Mar. 27, 2018

Related U.S. Application Data
(60) Provisional application No. 62/477,325, filed on Mar. 27, 2017.

(51) Int. Cl. A61F 7/00

(52) U.S. Cl. CPC A61F 7/0085; A61F 2007/0086; A61F 2007/006; A61F 2007/0011; A61F 2007/0029

(57) ABSTRACT

An apparatus for applying cold-air to a body-part of a user; the apparatus includes a housing, a cooling-means, an air-conduit, an application-means, an air-distribution means and a power-supply. The apparatus is useful for applying cold-air to a body-part of a user to soothe and reduce swelling and inflammation to promote healing and comfort.

### CROSS REFERENCE TO RELATED APPLICATION

The present application is related to and claims priority to U.S. Provisional Patent Application No. 62/477,325 filed Mar. 27, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of cold therapy and more specifically relates to an apparatus for applying cold-air to a body-part of a user.

### 2. Description of Related Art

The application of hot or cold to localized pain such as muscle or tendon pain to reduce swelling has a long history. There are many devices for heating or cooling parts of the body. Hot water bottles and ice or cold packs are among the oldest and simplest devices for applying heat and cooling respectively. Many doctors and therapists recommend applying a cold compress or ice pack to an injury in order to decrease recovery time. Placing conventional ice packs on a body part is often uncomfortable, if not unbearable for the user. The ice packs do not allow the skin underneath to breathe and are often too cold. They also have a tendency to melt or ‘go warm’ quickly. This requires the user to refreeze the pack and wait in order to continue icing. It can also be difficult for individuals to ice their body while asleep due to the constant need to replace the ice packs and the uncomfortable sensation. An effective alternative is needed.

U.S. Pat. No. 5,948,012 to Mark V. Mahaffey relates to a cold therapy device. The described cold therapy device includes a small portable ice chest and an electronic control unit to provide a unit which is safe to use right after surgery and has a portion which may be taken home by the patient. The combined unit provides the safety and accuracy of an institutional unit and the practicality and affordability of a portable take home unit. The cold therapy device includes an electronic control unit similar to the current institutional units in that it includes a thermoelectric module along with its associated electronic controls and a pump. The electronic control unit includes couplings to connect hoses leading to the patient and the cooling pad. Further, the portable ice chest having a lid fitted with an electric fluid pump which includes quick connect couplings allowing the ice chest to be placed in fluid communication with the electronic control unit. Finally, the fluid pump is electrically connected to the electronic control unit.

### BRIEF SUMMARY OF THE INVENTION

In view of the foregoing disadvantages inherent in the known cold therapy art, the present disclosure provides a novel body-part cooling apparatus. The general purpose of the present disclosure, which will be described subsequently in greater detail, is to provide cold-air to a body-part of a user to soothe and reduce swelling and inflammation.

An apparatus is disclosed herein. The apparatus includes a housing and a cooling-means disposed within the housing. The cooling-means may be configured to produce and move cold-air. An air-conduit may also be provided, and include a first-end, a second-end opposite the first-end, and a conduit-length therebetween, one of the first-end and the second-end being coupled to the housing.

Further, an application-means may be removably coupled to another one of the first-end and the second-end of the air-conduit and include an air-outlet. The application-means may be configured to apply the cold-air to the body-part of the user. In addition to this, an air-distribution means may be disposed within the housing and configured to distribute the cold-air out through the air-outlet via the air-conduit. A power-supply may also be connected to the housing and configured to provide power to the cooling-means and the air-distribution means.

A method of using the apparatus is also disclosed herein. The method of using the apparatus may comprise the steps of: providing the apparatus as above; attaching an application-means to the one of the first-end and the second-end of the air-conduit; turning the power-supply on to the cooling-means and the air-distribution means; and placing the application-means over the body-part of the user. Removal of the apparatus is performed when desired.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a body-part cooling apparatus, constructed and operative according to the teachings of the present disclosure.

FIG. 1 is a front perspective view of the apparatus during an ‘in-use’ condition, according to an embodiment of the disclosure.

FIG. 2 is a front perspective view of the apparatus of FIG. 1, according to an embodiment of the present disclosure.

FIG. 3 is a front perspective view of the apparatus of FIG. 1, according to an embodiment of the present disclosure.

FIG. 4 is a front perspective view of the apparatus of FIG. 1, according to an embodiment of the present disclosure.

FIG. 5 is a flow diagram illustrating a method of use for the disclosed apparatus, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to cold therapy and more particularly to a body-part cooling apparatus as used to improve the means for applying cold-air to a body-part of a user.

Generally disclosed is a cooling system for injured body parts that may replace a need for conventional ice packs by offering a cooling option for use with swollen or injured areas of a body that does not apply pressure and enables the injured area to ‘breathe’. The system may cool the body without melting and without the need to be frozen, allowing users to apply a cold compress to their body while sleeping with the use of a timer. The system may enable users to cool their injury comfortably and conveniently, increasing recovery rates.

Further, the system may be designed to cool swollen body parts and assist in healing injuries. The system may include a device comprised of a small plastic box containing a cooling device, a fan, a small insulated hose, a thermostat, and multiple outlet devices. The device may be plugged in, and ice-cold air may blow through the insulated hose to an attachment that is placed either beside, in front of, or under a sore/swollen body part. The thermostat may raise or lower the cooling to a suitable temperature, and a timer may start and stop the cold air. The cooling pads/attachments may be available in three options-a bendable pad, a rigid pad, and a flat pad for the back. The exact specifications may vary.

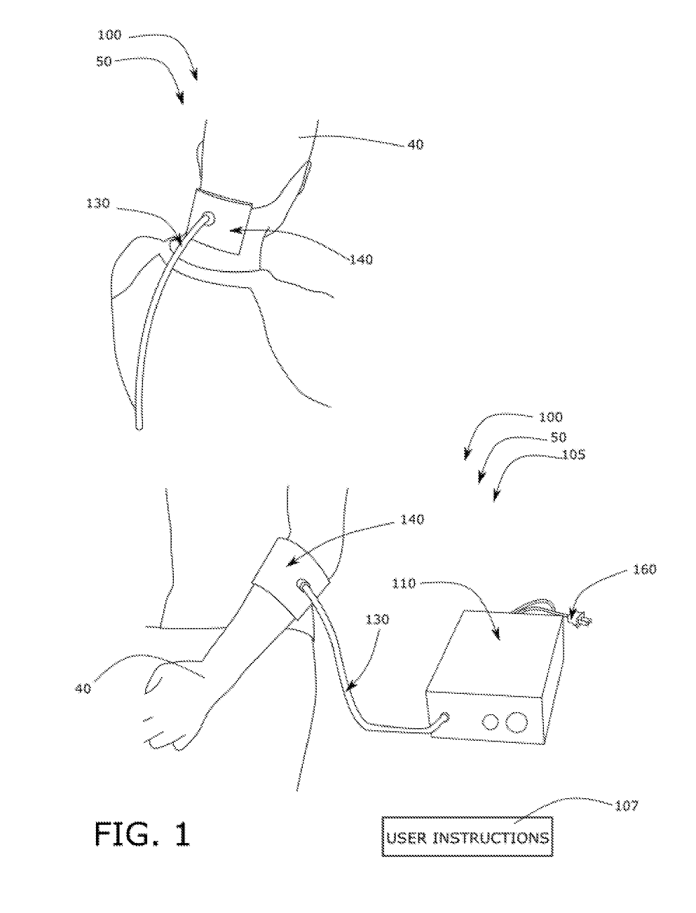

Referring now more specifically to the drawings by numerals of reference, there is shown in FIGS. 1-4, various views of an apparatus 100.

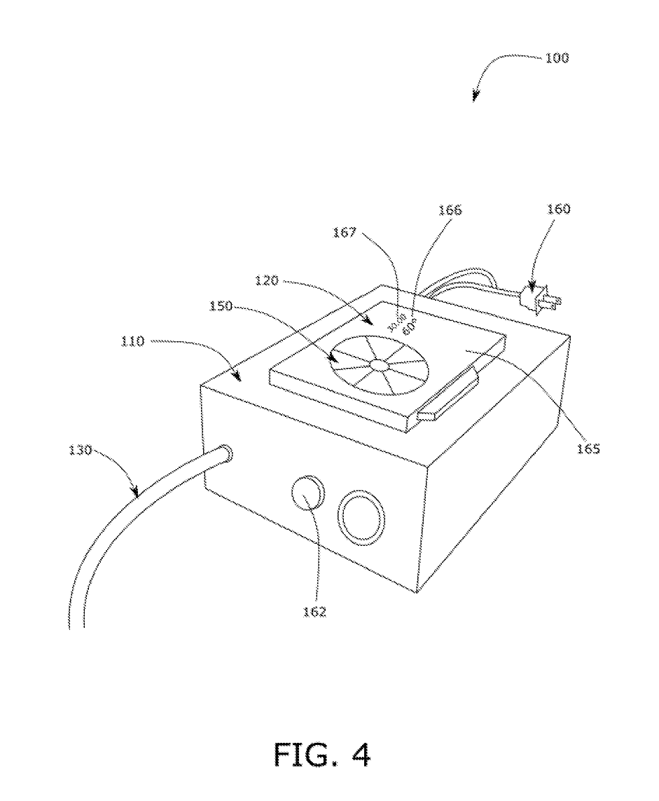

FIG. 1 shows the apparatus 100 during an ‘in-use’ condition 150, according to an embodiment of the present disclosure. As illustrated, the apparatus 100 may include a housing 110, a cooling-means 120 (FIG. 4), an air-conduit 130, an application-means 140, an air-distribution means 150 (FIG. 4) and a power-supply 160. In a preferred embodiment, the housing 110 may be made from a plastic-material. However, it should be appreciated that other suitable materials may be used.

According to one embodiment, the apparatus 100 may be arranged as a kit. The kit 105 may include set of user instructions 107. The instructions 107 may detail functional relationships in relation to the structure of the apparatus 100 (such that the apparatus 100 can be used, maintained, or the like, in a preferred manner).

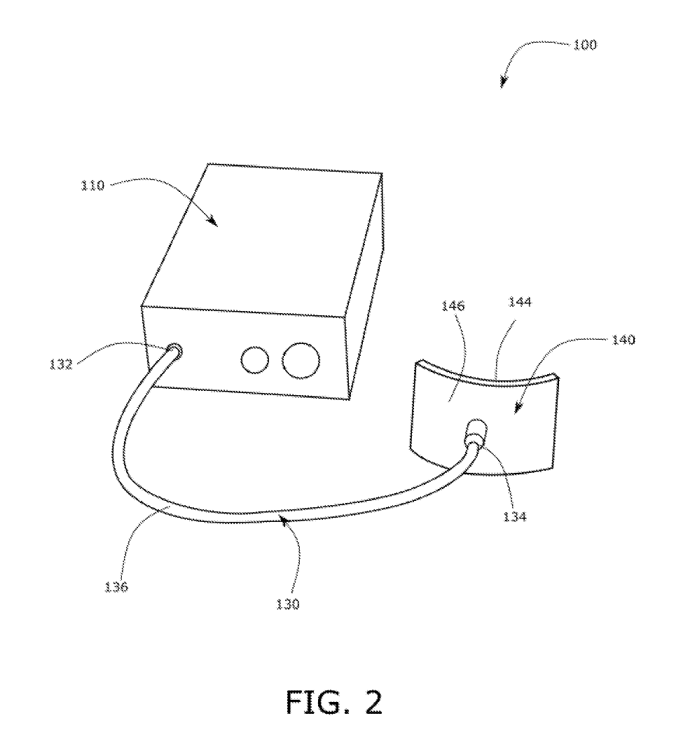

Referring now to FIG. 2 showing a front perspective view of the apparatus 100 of FIG. 1, according to an embodiment of the present disclosure. As shown, the air-conduit 130 may include a first-end 132, a second-end 134 opposite the first-end 132, and a conduit-length 136 therebetween and one of the first-end 132 and the second-end 134 being coupled to the housing 110. The air-conduit 130 may include an insulated hose. Further, in a preferred embodiment, the air-conduit 130 may be made from a rubber-material. In addition to this, the application-means 140 may be removably coupled to another one of the first-end 132 and the second-end 134 of the air-conduit 130. The application-means 140 may be a pad including a front-surface 144 and a rear-surface 146, and as shown, the rear-surface 146 may be configured for attachment to one of the first-end 132 and the second-end 134 of the air-conduit 130.

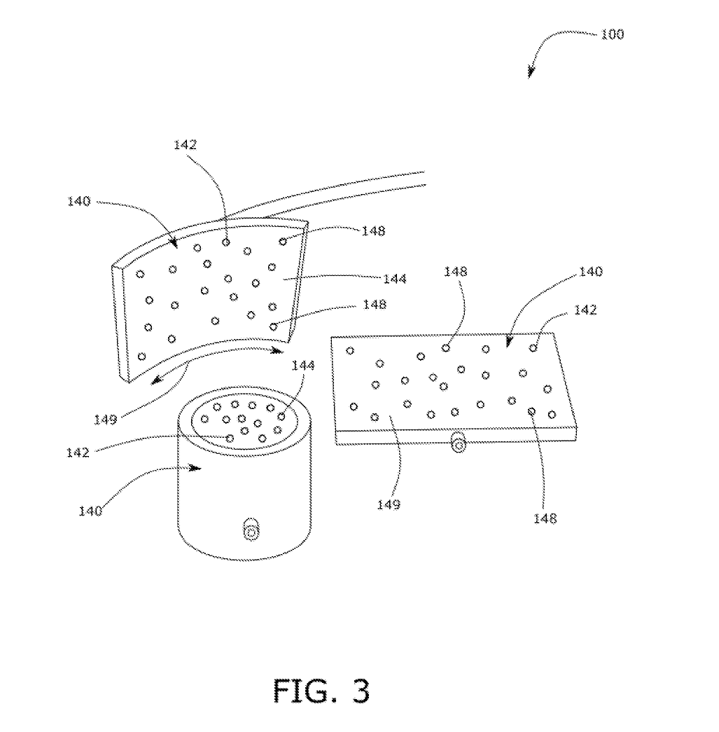

FIG. 3 shows a front perspective view of the apparatus 100 of FIG. 1, according to an embodiment of the present disclosure. As shown in this figure, the application-means 140 may include an air-outlet 142 and the application-means 140 being configured to apply the cold-air to the body-part 5 of the user 10 (FIG. 1). Further to this, the front-surface 144 may include the air-outlet 142 and may be configured for placement over the body-part 5 of the user 10. In addition, as shown, the air-outlet 142 may be a plurality of air-apertures 148. However, in other embodiments, the air-outlet 142 may be one air-aperture.

The front-surface 144 of the application-means 140 may include a curvature 149 shaped to accommodate the body-part 5 of the user 10. As shown, the application-means 140 may be interchangeable. In the preferred embodiment, the apparatus 100 may be provided with a plurality of application-means 140 each sized for placement over a particular body-part 5 of the user 10. For example, one application-means 140 may be sized for placement over an arm of the user 10, and another application-means 140 may be sized for placement over a back of a neck of the user 10 (as shown in FIG. 1).

Referring now to FIG. 4 showing a front perspective view of the apparatus 100 of FIG. 1, according to an embodiment of the present disclosure. As demonstrated, the cooling-means 120 may be disposed within the housing 110 and configured to produce the cold-air. In a preferred embodiment, the cooling-means 120 may be an air-conditioning unit (or other cooling means) sized to fit within the housing 110. Cooling may be achieved through a refrigeration cycle, evaporation, free cooling, or the like. Further, the air-distribution means 150 is preferably disposed within the housing 110; the air-distribution means 150 configured to distribute the cold-air out through the air-outlet 142 via the air-conduit 130. In the preferred embodiment, as shown, the air-distribution means 150 may be a fan. A combination of the cooling-means 120 and the air-distribution means 150 may distribute conditioned, cold-air out through the air-conduit 130.

The apparatus 100 may further include a control-circuit 165, a thermostat 166 and a timer 167. The thermostat 166 may be communicably coupled to the control-circuit 165 and configured to sense a temperature of the cooling-means 120. This may allow the temperature to be maintained at a desired setting. Further, the timer 167 may be communicably coupled to the control-circuit 165 and include a plurality of timer-settings. For example, one of the plurality of timer-settings may be a 30-minute timer 167. In this example, the user 10 may select the 30-minute timer 167 to apply the cold-air to the body-part 5 whilst the user 10 sleeps or performs other tasks without having to switch the apparatus 100 off. In addition, in one embodiment, when the 30-minute timer 167 has elapsed, the timer 167 may sound an alarm. The alarm may be an audible chime, a vibration, or the like.

The power-supply 160 may connected to the housing 110 and the power-supply 160 may configured to provide power to the cooling-means 120 and the air-distribution means 150. In addition to this, as shown, the power-button 162 may located on an outer-surface of the housing 110 and configured to selectively turn the power-supply 160 on and off. Other inputter means may be used.

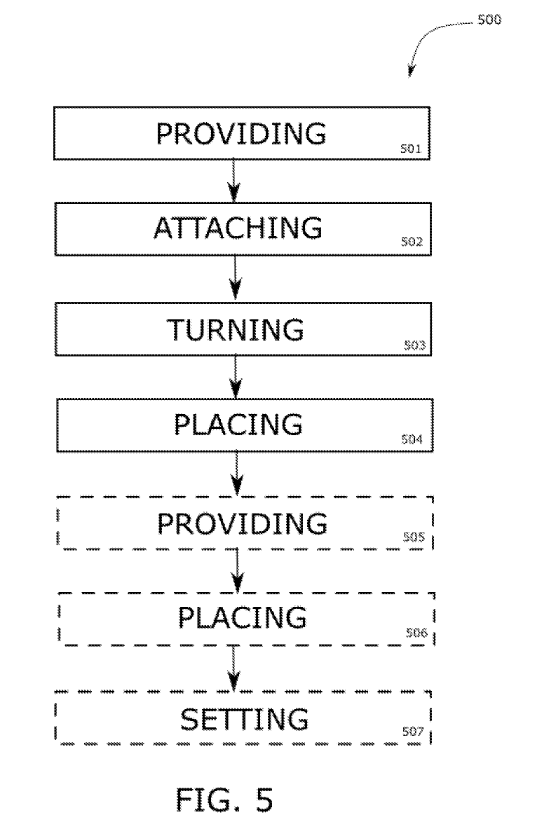

Referring now to FIG. 5, a flow diagram illustrating a method of using an apparatus for applying cold-air to a body-part of a user 500, according to an embodiment of the present disclosure. As illustrated, the method of using an apparatus for applying cold-air to a body-part of a user 500 may include the steps of: step one 501, providing the apparatus 100 as above; step two 502, attaching an application-means 140 to the one of the first-end 132 and the second-end 134 of the air-conduit 130; step three 503, turning the power-supply 160 on to the cooling-means 120 and the air-distribution means 150; and step four 504, placing the application-means 140 over the body-part 5 of the user 10. Further steps may include: step five 505, providing the apparatus 100 further including a timer 167 communicably-coupled to the cooling-means 120 and the air-distribution means 150; step six 506, placing the application-means 140 over the body-part 5 of the user 10; and step seven 507, setting a predetermined time-setting 168 on the timer 167.

It should be noted that step five 505, step six 506 and step seven 507 are optional steps and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for apparatus 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An apparatus for applying cold-air to a body-part of a user, the apparatus comprising:
a housing;
a cooling-means disposed within the housing, the cooling-means configured to produce the cold-air;
an air-conduit including a first-end, a second-end opposite the first-end, and a conduit-length therebetween, one of the first-end and the second-end being coupled to the housing;
an application-means removably coupled to another one of the first-end and the second-end of the air-conduit, the application-means including an air-outlet, and the application-means being configured to apply the cold-air to the body-part of the user;
an air-distribution means disposed within the housing, the air-distribution means configured to distribute the cold-air out through the air-outlet via the air-conduit; and
a power-supply connected to the housing, the power-supply configured to provide power to the cooling-means and the air-distribution means.

2. The apparatus of claim 1, wherein the cooling-means is an air-conditioning unit sized to fit within the housing.

3. The apparatus of claim 1, wherein the air-distribution means is a fan.

4. The apparatus of claim 1, wherein the application-means is a pad including a front-surface and a rear-surface.

5. The apparatus of claim 4, wherein the rear-surface is configured for attachment to one of the first-end and the second-end of the air-conduit.

6. The apparatus of claim 5, wherein the front-surface includes the air-outlet and is configured for placement over the body-part of the user.

7. The apparatus of claim 6, wherein the air-outlet is a plurality of air-apertures.

8. The apparatus of claim 7, wherein the front-surface of the application-means includes a curvature shaped to accommodate the body-part of the user.

9. The apparatus of claim 1, further comprising a control-circuit.

10. The apparatus of claim 9, further comprising a thermostat communicably coupled to the control-circuit and configured to sense a temperature of the cooling-means.

11. The apparatus of claim 10, further comprising a timer communicably coupled to the control-circuit.

12. The apparatus of claim 11, wherein the timer includes a plurality of timer-settings.

13. The apparatus of claim 1, further comprising a power-button located on an outer-surface of the housing and configured to selectively turn the power-supply on and off.

14. The apparatus of claim 1, wherein the air-conduit includes an insulated hose.

15. The apparatus of claim 1, wherein the application-means is interchangeable.

16. The apparatus of claim 15, wherein the housing is made from the plastic-material.

17. An apparatus for applying cold-air to a body-part of a user, the apparatus comprising:
a housing;
a cooling-means disposed within the housing, the cooling-means configured to produce the cold-air;
an air-conduit including a first-end, a second-end opposite the first-end, and a conduit-length therebetween, one of the first-end and the second-end being coupled to the housing;
an application-means removably coupled to another one of the first-end and the second-end of the air-conduit, the application-means including an air-outlet, and the application-means being configured to apply the cold-air to the body-part of the user;
an air-distribution means disposed within the housing, the air-distribution means configured to distribute the cold-air out through the air-outlet via the air-conduit;
a power-supply connected to the housing, the power-supply configured to provide power to the cooling-means and the air-distribution means;
a control-circuit;
a thermostat communicably coupled to the control-circuit and configured to sense a temperature of the cooling-means;
a timer communicably coupled to the control-circuit;
a power-button located on an outer-surface of the housing and configured to selectively turn the power-supply on and off; and
wherein the cooling-means is an air-conditioning unit sized to fit within the housing;
wherein the air-distribution means is a fan;
wherein the application-means is a pad including a front-surface and a rear-surface;
wherein the rear-surface is configured for attachment to one of the first-end and the second-end of the air-conduit;
wherein the front-surface includes the air-outlet and is configured for placement over the body-part of the user;
wherein the air-outlet is a plurality of air-apertures;
wherein the front-surface of the application-means includes a curvature shaped to accommodate the body-part of the user;
wherein the timer includes a plurality of timer-settings;
wherein the air-conduit includes an insulated hose;
wherein the application-means is interchangeable; and
wherein the housing is made from a plastic-material.

18. The apparatus of claim 17, further comprising set of instructions; and
wherein the apparatus is arranged as a kit.

19. A method of using an apparatus for applying cold-air to a body-part of a user, the method comprising the steps of:
providing the apparatus for applying cold-air to a body-part of a user having;
a housing;
a cooling-means disposed within the housing, the cooling-means configured to produce the cold-air;
an air-conduit including a first-end, a second-end opposite the first-end, and a conduit-length therebetween, one of the first-end and the second-end being coupled to the housing;
an application-means removably coupled to another one of the first-end and the second-end of the air-conduit, the application-means including an air-outlet, and the application-means being configured to apply the cold-air to the body-part of the user;
an air-distribution means disposed within the housing, the air-distribution means configured to distribute the cold-air out through the air-outlet via the air-conduit; and
a power-supply connected to the housing, the power-supply configured to provide power to the cooling-means and the air-distribution means;
attaching an application-means to the one of the first-end and the second-end of the air-conduit;
turning the power-supply on to the cooling-means and the air-distribution means; and
placing the application-means over the body-part of the user.

20. The method of claim 19, further comprising the steps of:
providing the apparatus further including a timer communicably-coupled to the cooling-means and the air-distribution means;
placing the application-means over the body-part of the user; and
setting a predetermined time-setting on the timer.
